(19) United States
(12) Patent Application Publication
(10) Pub. No.: US 2010/0240959 A1
(43) Pub. Date: Sep. 23, 2010

(54) RETRACTOR AND SEALING SYSTEM FOR SURGICAL/NON-SURGICAL INSTRUMENTS

(76) Inventor: John R. Donahue, King of Prussia, PA (US)

Correspondence Address: VOLPE AND KOENIG, P.C., UNITED PLAZA, 30 SOUTH 17TH STREET, PHILADELPHIA, PA, 19103, US

(21) Appl. No.: 12/795,055

(22) Filed: Jun. 7, 2010

Related U.S. Application Data
(63) Continuation of application No. PCT/US2008/085645, filed on Dec. 5, 2008.
(60) Provisional application No. 60/992,402, filed on Dec. 5, 2007.

(51) Int. Cl. A61B 1/32

(52) U.S. Cl. 600/204

(57) ABSTRACT

A universal retractor and sealing system used in conjunction with surgical instruments such as cannulas, endoscopes surgical or non-surgical instruments or other tools. The user manipulates the instrument so as to create a projecting surface located inside the cavity. An outward force applied to the instrument body seats the projecting surface against the inner wall of the cavity, which can be used to form at least a partial seal against fluid. With the projecting surface positioned against the cavity wall, the instrument can be manipulated so as to act as a retractor, expanding the workable area and/or the field of view as required.

### CROSS REFERENCE TO RELATED APPLICATIONS

This application is a continuation of PCT/EP2008/085645, filed Dec. 5, 2008, which claims the benefit of U.S. Provisional Application No. 60/992,402, filed Dec. 5, 2007, which are incorporated herein by reference as if fully set forth.

### BACKGROUND

The present invention is directed to a universal retractor and sealing system for use as a stand alone instrument or for use with cannulas, endoscopes and other medical tools typically used in minimally invasive surgery, designed to maintain, seal, and manipulate surgical incisions in tissue, as well as for non-medical tools that can be used in various applications.

Modern surgical procedures frequently require the use of cannulas inserted into an incision to provide a means of introducing surgical instruments into the body cavity. In arthroscopic or endoscopic procedures, the cavity is often filled with fluid, which can be a liquid or gas, and kept under moderate pressure, requiring these cannulas to have a firm fit in the surrounding tissue to prevent the loss of these fluids. However, there is generally some fluid leakage into the surrounding tissue which causes swelling of tissue, both outwardly as well as into the site of the surgery, reducing the room for manipulation of instruments and further limiting the field of view. This is particularly problematic in the knee and shoulder surgery where there is generally less than 1 cm of space for both visualizing and manipulating surgical tools in the affected area of the joint or other tissue, and often less than 5 mm of space between the cannula distal end and the affected area of cartilage or tissue that severely limits the possible field of view of an endoscope inserted through the cannula by which the surgeon visualizes the site and the actual cartilage or tissue being removed, smoothed, or repaired.

Several methods of fixing and sealing cannulas to incisions are currently in use today. For example, U.S. Pat. No. 5,697,913 discloses the use of a stepped cannula designed to engage the surrounding tissue. Similarly, U.S. Pat. No. 5,364,372 shows the use of a threaded surface as a means to seal and affix the cannula to tissue. In addition to offering limited sealing ability, experience has shown screw and step-type cannulas still do not hold in position in the surrounding tissue. They also allow increased swelling due to fluid seepage into the tissue further limiting the field of view due to inward swelling, as well as causing elongated recovery times for patients. Further, these types of cannulas are easily dislodged during the course of surgery adding unnecessary delay to the procedure.

### SUMMARY

The present invention is directed to a universal retractor and sealing system for use as a stand-alone instrument or for use with cannulas, endoscopes and other surgical and non-surgical tools. A sleeve having a distal end section that is expandable or includes an expandable portion is connected to or formed with the instrument and movable with respect thereto. The instrument and sleeve are insertable into a surgical opening where the user manipulates the sleeve so as to expand the distal end section to increase the sleeve diameter creating at least a partial projecting surface inside the operating cavity. An outward force applied to the instrument body seats the projecting surface against the tissue, forming at least a partial seal to prevent or hinder bodily and surgical fluids from escaping into the surrounding tissue. Once inserted, the system provides for significantly improved sealing to reduce swelling and damage to the surrounding tissue, while preventing the possibility of accidental dislodge. Further, with the projecting surface positioned against the cavity wall, the instrument can be used as a retractor, expanding the operating area as required, including increasing the viewing field available for an endoscope.

In a preferred embodiment, the retractor and sealing system is provided with a cannula. However, it can be provided with an endoscope, or in another aspect of the invention is easily retrofitted to existing endoscopes or any other instrument or tool.

### BRIEF DESCRIPTION OF THE DRAWINGS

The present invention will be explained in more detail in connection with the drawings in which presently preferred embodiments are shown.

In the drawings:

FIG. 1 is a perspective view of a first embodiment of a system according to the present invention with the projecting surfaces in their installation state.

FIG. 1A is a top view of the first embodiment of the invention shown in FIG. 1

FIG. 1B is a bottom view of the first embodiment of FIG. 1

FIG. 2 shows a second embodiment of the invention featuring the device used with an existing cannula.

FIGS. 3, 3A and 3B show a third embodiment of a system according to the present invention featuring projecting surfaces deployed by a rotatable collar located at the proximal end of the instrument including cross-sectional views taking along lines 3A-3A and 3B-3B.

FIGS. 4 and 4A are views of a fourth embodiment of the present invention featuring an obturator with a split nose, shown in the installation state and in the deployed state.

FIGS. 5-8 show the fifth embodiment of the present invention featuring an outer sleeve with folding seams in transition from the installation state to a deployed state.

FIG. 8A is the bottom view of the embodiment found in FIG. 8 with the projecting surface comprising a plurality of fingers.

FIG. 8B is a bottom view of an alternate fifth embodiment of the invention with the projecting surface forming a continuous annular shape.

FIG. 9 is a cross-section of the body cavity with an embodiment of the present invention inserted therein.

FIG. 10 is a perspective view of a sixth embodiment of the present invention featuring a lock to hold the outer sleeve at varying positions with respect to the body.

FIGS. 11 and 11A show a seventh embodiment of the present invention integrated into an endoscope in the installation state and the deployed state, respectively.

FIGS. 12 and 12A show an eighth embodiment of the present invention featuring a universal version of the invention applied to an endoscope in the installation state and deployed state.

FIG. 13 is a ninth embodiment of the present invention featuring a projecting surface comprised of a deformable bladder.

FIG. 14 is a view of a tenth embodiment of a cannula according to the present invention.

FIG. 15 is a cross-sectional view of an eleventh embodiment of an instrument according to the present invention.

FIGS. 16 and 16A are views of a twelfth embodiment of a cannula according to the present invention.

FIGS. 17A and 17B are views of a cannula with a retractor and sealing assembly according to a thirteenth embodiment of the present invention.

FIG. 18 is a detailed view showing one leg of the retractor and sealing assembly of FIGS. 17A and 17B.

FIG. 19 is the view of the top of a cannula with a cap according to the present invention.

FIG. 20 is a top view of the cap.

FIG. 21 is a half cross-sectional view through the cap.

FIG. 22 is a side view of the cap.

FIG. 23 is a view similar to FIG. 13, showing the cannula with additional seals and a removable and adjustable outer holding member.

FIG. 24 is a top view of the holding member shown in FIG. 23.

FIG. 25 is a cross-sectional view through a cannula having additional external sealing members attached thereto that prevent or control fluid coming out through the cannula.

FIG. 26 is a top view of the sealing member of FIG. 25.

FIG. 27 is a view similar to FIG. 13, showing the cannula with a deformable bladder and a port on the cannula body for connection to a syringe for inflating the bladder.

### DETAILED DESCRIPTION OF THE PREFERRED EMBODIMENTS

Certain terminology is used in the following description for convenience only and is not considered limiting. Words such as “front”, “back”, “top” and “bottom” designate directions in the drawings to which reference is made. This terminology includes the words specifically noted above, derivatives thereof and words of similar import. Additionally, the terms “a” and “one” are defined as including one or more of the referenced item unless specifically noted.

The preferred embodiments of the present invention will be described with reference to the drawing figures where like numerals represent like elements throughout.

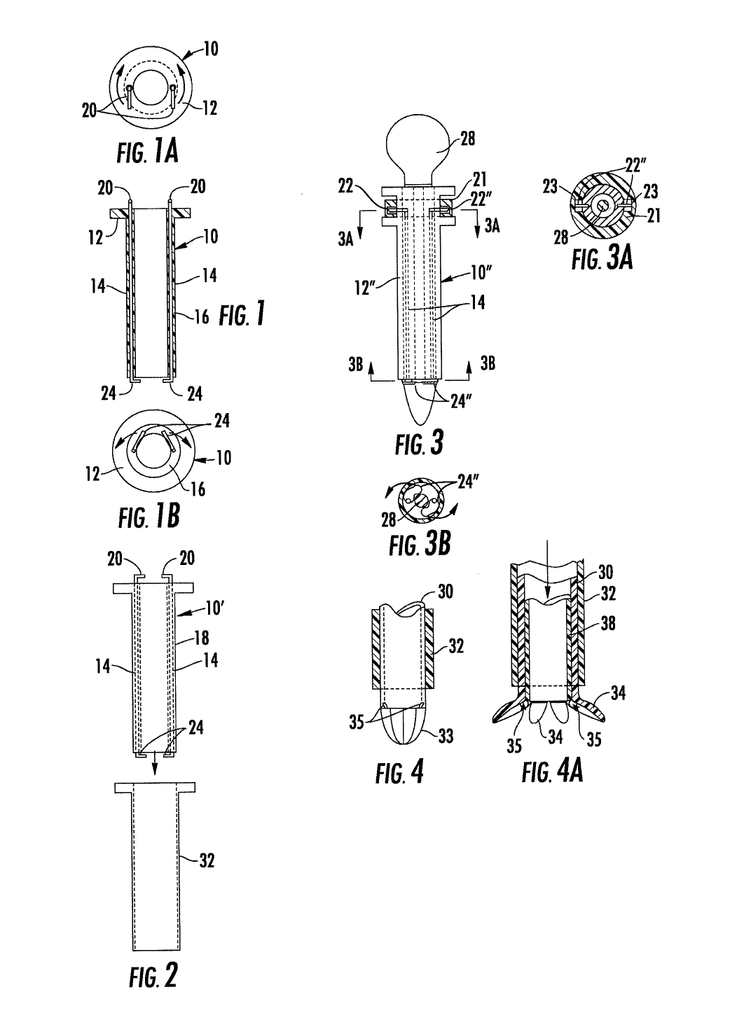

Referring to FIG. 1, a first preferred embodiment of the invention in the form of a cannula 10 is shown, having an instrument body 12 in which opposing drive pins 14 are located in longitudinally extending openings defined in the side wall 16. While the side wall 16 is shown smooth, those skilled in the art will recognize that it can have a contoured outer surface, with for example, threads or annular bumps typically used on cannulas. Handles 20 are provided at the proximal ends of the pins 14, which via a rotational force on handles 20, rotate projecting surfaces 24 located at the distal ends of the pins 14 from a stowed position to a projecting position. FIGS. 1A and 1B show both proximal and distal ends respectively, with the handles 20 and the projecting surfaces 24 oriented for installation. Projecting surfaces 24 remain oriented inward toward the axis of the body 10 so as to allow for easy insertion into the incision. The rotation of the handles 20 in the direction shown in FIG. 1A results in the rotation of the projecting surfaces 24 into their deployed state, extending radially outward, facilitating use of the cannula 10 as a retractor. In the deployed state, the opening defined through the cannula body 12 is free for the insertion of an endoscope or other instrument. The projecting surfaces 24, once deployed, hold the cannula in position, and also allow the cannula to be used as a retractor. While two pins 14 are shown, there can be three, four or more pins, preferably spaced apart equidistantly around a circumference of the body 12. To the extent that these projecting surfaces 24 hold the cannula 10 in position in a more firm manner, they also assist sealing of the cannula 10 in the tissue at the operating site.

Preferably, the cannula body 12 is made of a medically suitable polymeric material, and the pins 14 are made of a suitable medical grade metal, such as stainless steel. Those skilled in the art will also recognize that the pins 14 and/or body 12 could have interfacing ramped surfaces so that the projecting surfaces 24 would be moved downwardly and outwardly as they are deployed by turning the handles 20.

While the cannula embodiment has been described, this can also be utilized in endoscopes and other medical or non-medical instruments.

Referring to FIG. 2, a second embodiment of the invention is shown that can be used with an existing cannula 32. In this embodiment, the retractor and sealing assembly 10′ is provided as a separate part in which the drive pins 14 with the associated handles 20 and projecting surfaces 24 are located in longitudinally extending openings in a separate sleeve 18 that can be inserted into the cannula 32.

Once inserted, the handles 20 can be rotated to deploy the projecting surfaces 24 so that the cannula 32 in combination with the retractor and sealing assembly 10′ can be used as a retractor, and in view of the additional position holding ability provided, also assists in sealing the cannula.

In a third embodiment of the present invention show in FIGS. 3-3B, a cannula 10″, similar to the cannula 10 described above, is provided. Here, the obturator 28 is also shown in FIG. 3 that is used to insert the cannula 10″. In this embodiment, the cannula 10″ includes a body 12″ with a rotatable collar 21 at the proximal end that engages the actuating elements 22″ in slots 23, shown in FIG. 3A, so that rotation of the collar 21 turns pins 14 to deploy the projecting surfaces 24″ through a rotational force transmitted through drive pins 14. Cross-sectional views 3A and 3B show the engagement between the rotatable collar 21 and the actuating elements 22″, and the direction traveled by the projecting surfaces 24″ as they move from the installation state to the deployed state.

This embodiment of the invention provides the same advantages of the prior embodiments, and allows more convenient deployment of the projecting surfaces 24″ in a single manipulation, via rotation of the collar 21. Those skilled in the art will understand from the present disclosure that the deployment could also use various other types of rotating, sliding or projecting mechanisms extending in, through or on the outside of the body of the instrument.

FIG. 4 shows a fourth embodiment of the present invention in which the retractor and sealing system are configured into the obturator 30, which can be inserted through a cannula 32, a portion of which is shown. The obturator 30 includes a split nose 33 and deploying projections 35 located around an inner periphery of the split nose in proximity to the ends of the split lines. As shown in FIG. 4A, a sleeve 38 is inserted into the obturator 30 and is pressed downward, so that the distal end of the sleeve 38 contacts the deploying arms 35, which in turn expand the split nose 33 so that projecting surfaces 34 are folded outwardly.

Preferably, the obturator 30 is formed from a medical grade polymeric material, and the split lines at the nose 33 are formed as weakened or perforated sections. Once deployed, the projecting surfaces 34 assist in holding and sealing the cannula 32 in position in the tissue through which it is inserted. Additionally, the projecting surfaces 34 allow the obturator 30-cannula 32 assembly to be used as a retractor.

FIGS. 5-9 show a fifth embodiment of the present invention in the form of a cannula 50 that is inserted using an obturator 52 that is led into the proper position for insertion by passing over a guide wire 54. The guide wire is used to aid the surgeon in guiding the instrument into close proximity with the operating site, and can be withdrawn, along with the obturator 52 after the cannula 50 is positioned.

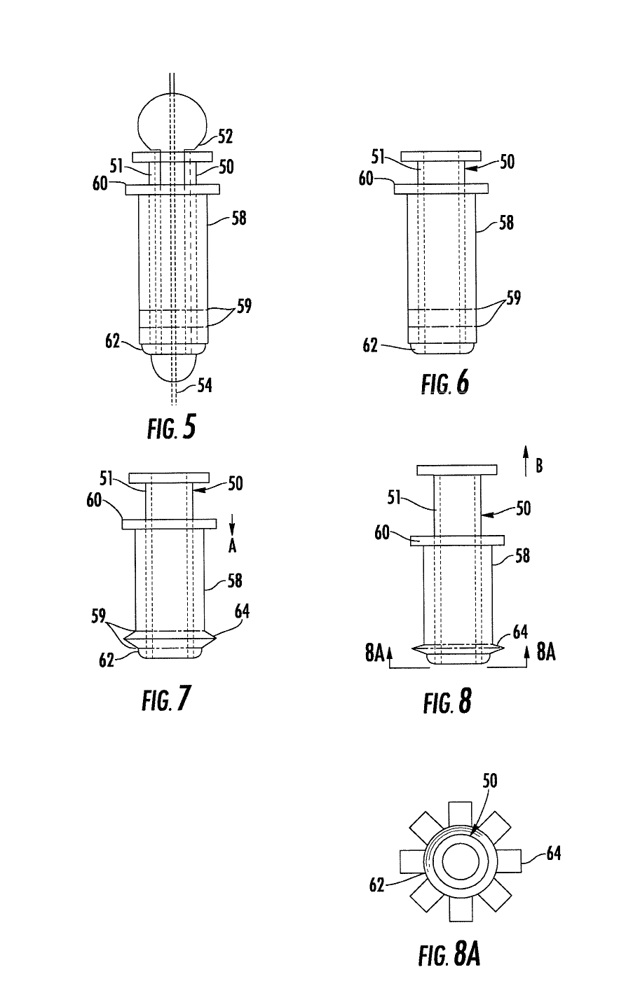

In accordance with the fifth preferred embodiment, which is illustrated without the obturator 52 and guide wire 54 in FIG. 6, a sleeve 58 with fold seams 59 and handle 60 is located over the cannula body 51. The fold seams 59 can be formed by a weakened or perforated section of the sleeve 58. A stop 62 is located proximate to a distal end of the body 51. The stop 62 can be integrally formed with the body 51 or can be attached in a separate manufacturing step.

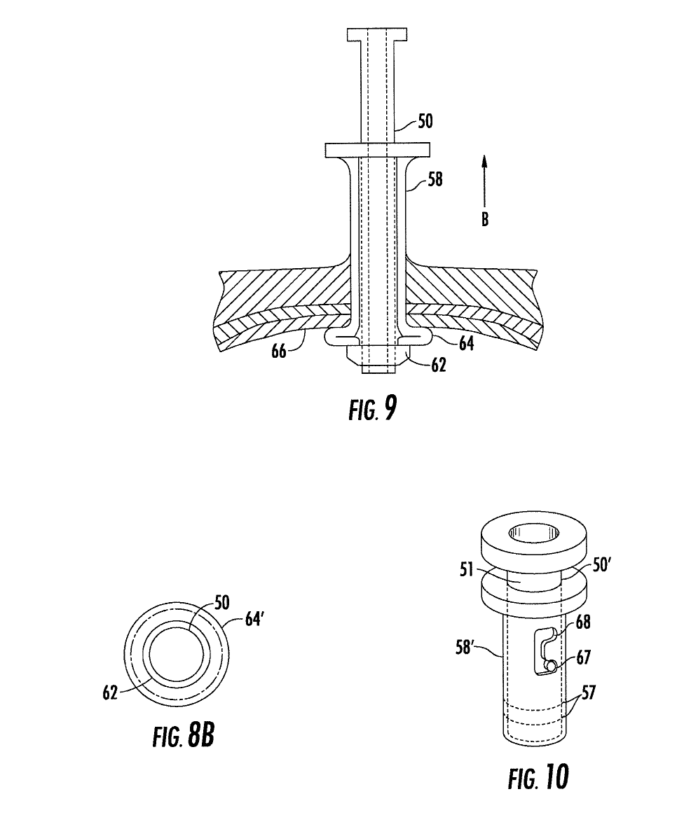

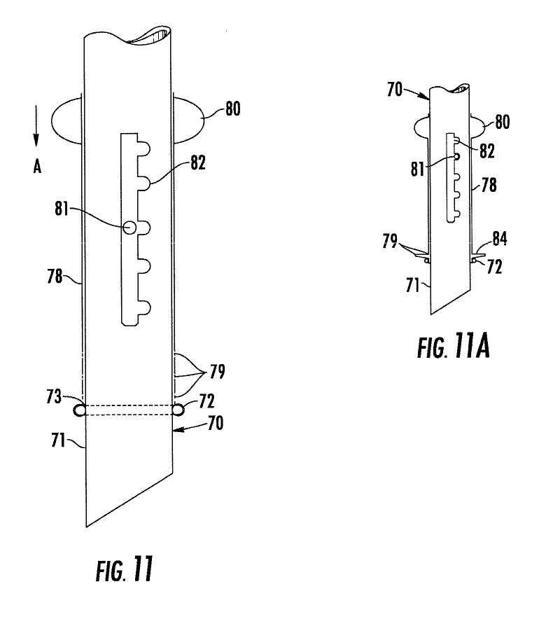

In the installation state, as shown in FIG. 6, projecting surfaces 64, which form along the fold seams 59 and can be deployed by an operator, are in a retracted position against the body 51, facilitating insertion of the cannula 50. Once the cannula 50 is inserted, the surgeon applies a downward force in the direction A, shown in FIG. 7, using the handle 60, forcing the sleeve 58 to slide toward the stop 62. As the sleeve 58 encounters the stop 62, the sleeve 58 is forced to yield at the fold seams 59, expanding in diameter as shown in FIG. 7. This expansion creates at least one projecting surface 64, and in this embodiment a plurality of projecting surfaces 64 as shown in FIG. 8A, which are formed inside the overlying tissue at the surgical site as depicted in FIG. 9. Once the projecting surfaces 64 have been formed, the cannula 50 can be pulled outward in direction B shown on FIG. 8, resulting in the projecting surfaces 64 contacting and forming a seal against the cavity wall.

Additionally, with the at least one projecting surface 64 in place, and preferably, with a plurality of projecting surfaces 64 that extend around the entire circumference of the cannula 50, it can be used as a retractor, offering control of the surrounding tissue to alter the available space found in the cavity. This offers an improved tissue sealing capability as well based on the configuration of the projecting surfaces 64, and prevents swelling tissue at the surgical site from swelling down over the end of the cannula 50. Further, the distal tip of the cannula 50 ends at the stop 62, and does not project further downward, which also increases the field of view in comparison with known cannulas that have an extended, tapering distal tip.

The sleeve 58 is preferably made from a medical grade polymeric material, as is the cannula body 51. While a plurality of circumferentially spaced apart projecting surfaces are shown, it is also possible to have a sufficiently flexible material at the distal end of the sleeve 58, so that upon deployment, a single annular projecting surface 64 is formed.

Alternatively, the sleeve 58 can include at least one projecting surface 64 pre-formed near the distal end from a memory-retaining, resilient material. The distal end of the sleeve 58 is fixed to the cannula body 51 at or near the distal end. To move the system into its installation state, the user applies tension on the handle 60 in direction B, stretching the sleeve 58, thereby deforming the projecting surface 64 into a retracted position close or adjacent to the body. This provides for easy insertion of the cannula 50. Once inserted, the handle 60 is released and the resilient material reforms the projecting surface 64 inside the cavity. The application of force in direction B on the cannula 50 seats the projecting surfaces 64 against the tissue, forming the necessary seal with the cavity wall. The cannula 50 can then be further manipulated for use as a retractor as discussed above.

Referring generally to FIG. 8A, a bottom view of FIG. 8 with the projecting surfaces 64 created is shown as a plurality of fingers 64. As the sleeve 58 is forced into contact with the stop 62, the fingers 64 are extended radially in an orientation generally perpendicular to the axis of the cannula body 51. Alternatively, the same arrangement is formed with the sleeve 58 having preformed projections that are retracted for insertion and which then resiliently return to the expanded position. The fingers 64 form the projecting surface to seal the instrument with respect to the cavity wall as well as provide the instrument with the ability to be used as a retractor. The projecting surface 64′ can also be a continuous annular in shape, as depicted in FIG. 8B, which is an alternate bottom view of the invention shown in FIG. 8.

Referring to FIG. 9, the cannula 50 is shown as it would appear inserted into the body cavity is shown. Once inserted and the projecting surface 64 is deployed, force on the instrument body 51 in the direction of B results in a seal between tissue 66 and the projecting surface 64. The instrument can act as a retractor through further upward force applied to the instrument body 51 in direction B.

In accordance with a sixth embodiment of the invention, the cannula 50′ can also include a locking mechanism as show in FIG. 10. The mechanism is comprised of a locking channel 68 located on the sleeve 58′ and a locking pin 67 attached to the body 51 of the cannula 50′. The user rotates the sleeve 58′ with respect to the body 51 to a position allowing for movement of the sleeve 58 in the axial direction with respect to the body 51. To lock the sleeve 58′ and the body 51, after pressing the sleeve 58 downwardly toward the stop 62, the user again rotates the sleeve 58′ with respect the body 51, engaging the pin 67 into the horizontally extending portion of the locking channel 68, fixing the sleeve in the desired position.

FIG. 11 shows a seventh embodiment of the present invention integrated into an endoscope, cannula or other instrument or tool 70. A stop 72 is attached to a distal end of the endoscope tube 71, which preferably includes a recess 73. The stop 72 is preferably formed by a medical grade elastomeric ring having a round or tapered form that sits in the recess 73. A sleeve 78, similar to the sleeve 58 described above, is located on the endoscope tube 71, and includes fold seams 79, similar to the fold seams 59.

Preferably, a locking pin 81 is fixed to the endoscope tube 71. The sleeve 78 includes a stepped locking channel 82. In use, a downward force is placed on the handle 80 in the direction A, forcing sleeve 78 against stop 72 causing the deployment of the projecting surfaces 84, as shown in FIG. 11A. The sleeve 78 can be locked into place by positioning the locking pin 81 into the locking channel 82.

In an eighth embodiment of the present invention show in FIG. 12, the system is retrofitted to an existing endoscope 70′ (shown) or can be added to any type of cannula or other instrument or tool. The sleeve 78 with folding seams 79 and handle 80 is fitted over the endoscope tube. The stop 72′ is attached onto the distal of the tube by a press or friction fit. FIG. 12A shows the embodiment of FIG. 12 in its deployed state with projection surface 84 formed after the application of a downward force in the direction A on sleeve 78, forcing the distal end into the stop 72.

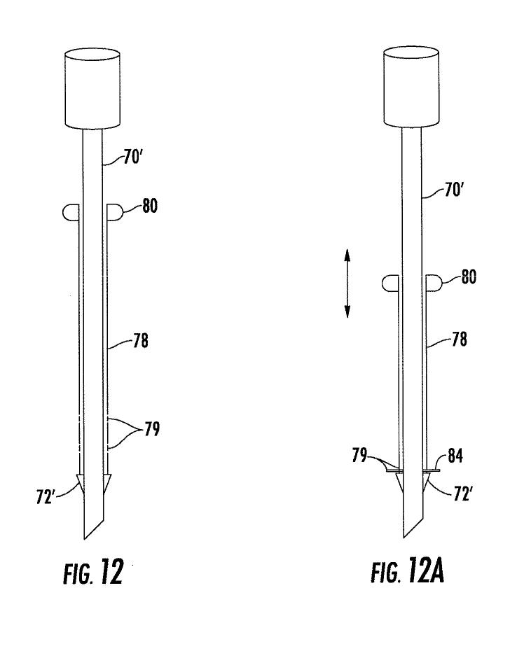

In a ninth embodiment shown in FIG. 13, which is shown in cross-section on the left side of the centerline, a cannula 90 is provided having a projecting surface formed by a bladder or elastic tube 92 attached to the distal end of the cannula and extending up the cannula body 91, where it is also attached at a proximal end. Preferably, a fluid or air is trapped between the cannula body 91 and the elastic tube 92. A slidable sleeve 93 is located over the bladder or elastic tube 92, and by pushing downwardly on the handle 94 of the sleeve 93, the trapped fluid is forced downwardly, creating an expanded projecting and sealing surface 95. By moving the sleeve 93 back toward the proximal end of the cannula body 91, the tube 92 elastically returns to its original position generally adjacent to the body 91 to allow for easy insertion and removal of the cannula 90. Alternatively, an outside fluid source can be utilized to adjust the expansion or contraction of the bladder or elastic tube 92.

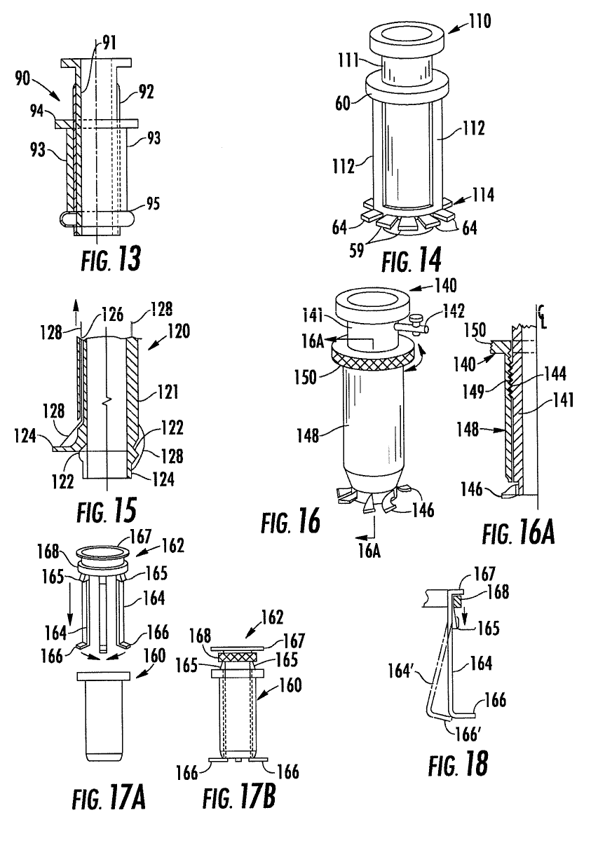

Referring to FIG. 14, a tenth embodiment of a cannula 110 according to the invention is shown. The cannula 110 is similar to the fifth embodiment of the cannula 50, except instead of the sleeve 58, only spaced apart strips 112 extend from the handle 60 to the distal end where a partial sleeve 114 is provided with the fold seams 59 (shown in the folded state). The projecting surfaces 64 formed along the folds are extended. Preferably, this is the neutral or rest position for the projecting surfaces 64, and in order to insert the cannula 110, the handle 60 is slid toward a proximal end of the cannula 110, so that tension carried through the strips 112 causes the projecting surfaces 64 to flatten out against the sides of the cannula body 111. Once inserted, the handle 60 is released and the projecting surfaces 64 return to the deployed position. However, those skilled in the art will recognize that this arrangement can also be configured such that the partial sleeve section 114 remains against the cannula body 111 in a neutral state and is deployed after insertion by pressing down on the handle 60 through a compressive force carried through appropriately sized strips 112.

Referring to FIG. 15, an eleventh embodiment of the instrument 120 according to the invention is shown in a split view in which the right half is shown with the distal end of the instrument in the pre-installation state and the left half is shown with the distal end of the instrument 120 in the installed and deployed state. The tubular body 121 is configured with a weakened hinge section 122 spaced apart from the distal end and is defined by a plurality of projecting surfaces 124 which are formed by separated segments of the distal end of the tubular body 121. The tubular body 121 includes a plurality of apertures 126 that extend longitudinally through the wall of the tubular body 121. Actuating wires 128 are located in these apertures 126 and the distal end of each actuating wire 128 is connected to a separate one of the projected surfaces 124. Preferably, openings are located in the sidewall of the body 121 near the distal end, above the hinged section 122, so that the actuating wires 128 extend outwardly from the body and are attached to the projecting surfaces 124. After insertion of the instrument 120, the actuating wires 128 are pulled, resulting in the projecting surfaces 124 being moved to the deployed position as shown in the left half of FIG. 15.

Based on the configuration of the body sidewall at the distal end of the tubular body 121, the projecting surfaces 124 can be limited in their travel path so that they are stopped generally perpendicular to the body 121 of the instrument 120 once deployed. This type of arrangement, as with all the prior embodiments, is applicable to cannulas or other surgical or non-surgical instruments. Additionally, those skilled in the art will appreciate that the projecting surfaces could be initially in a deployed state, and deflected using wires or guides to a stowed state for insertion and removal. Further, it is possible to use wires or guides to both deploy and stow the projecting surfaces, as required for a particular application.

Referring to FIGS. 16 and 16A, a twelfth embodiment of a cannula 140 according to the invention is shown. The cannula 140 includes a body 141 upon which a stopcock 142 can optionally be provided. As shown in FIG. 16A, preferably an external thread 144 is located on the body 141, preferably in proximity to the proximal end of the cannula 140. At the distal end of the body 141, a plurality of projecting surfaces 146, which are made from an elastically deformable material such as a shape memory allow or a suitable polymeric material, are attached to the body. These projecting surfaces 146 are formed such that in an un-deflected state, they project outwardly from the body 141 and can be used as a retractor or for sealing against tissue on the inside of the insertion site. A sleeve 148 is located over the body 141 and preferably has counter threads 149 which engage with the threads 144 on the body 141. A handle 150 is located at the proximal end of the sleeve 148. For insertion of the cannula 140, the sleeve 148 is advanced along the threads 144 on the body 141 such that the distal end of the sleeve 148 deflects the projecting surfaces 146 into a stowed position between the inner surface of the sleeve 148 and the distal end of the body 141. Once the cannula 140 is inserted, the sleeve 148 is rotated via the handle 150 in a preferably counter clockwise direction so that the projecting surfaces 146 are released from the stowed position and extend based on there elastic properties into the deployed position shown in order to provide a retractor and sealing surface.

Referring to FIGS. 17A and 17B, a thirteenth embodiment of a cannula 160 according to the invention is shown. According to the thirteenth embodiment of the invention, the cannula 160 can be any type of known cannula. In order to retrofit the known cannula 160 with a sealing and retracting assembly according to the invention, a separate seal/retractor assembly 162 is provided having a plurality of resilient legs 164 which terminate in retractor hooks 166 that extend generally perpendicular, as shown, or at any other angle to the legs 164. While three legs 164 are shown, those skilled in the art will recognize that more or less legs could be provided. Preferably, the retractor assembly 162 includes a proximal base with a handle 167. A ramp-shaped actuating surface 165 is located at the proximal end of each leg 164. An actuator ring 168 which can be grasped by a user is located above the actuating surfaces 165. In use, after the cannula 160 is inserted, if retraction is needed the legs 164 of a retractor assembly 162 are deflected inwardly by pressing down on the actuator ring 168 so that the legs 164 are deflected inwardly, as indicated by 164′ in FIG. 18, and can pass through the opening in the cannula 160. The retractor assembly 162 is then inserted and once the retractor hooks 166 clear the distal end of the cannula 160, the actuator ring 168 is slid upwardly and the legs 164 resiliently return to their original position such that the projecting surfaces provided by the hooks 166 project outwardly beyond the cannula 160 in order to form a retractor while still leaving a majority of the cannula opening to allow other instruments to be passed therethrough. The distal ends of the actuating surfaces 165 of the legs 164 can also act as a stop to set a maximum insertion depth of the retractor assembly 162, as shown in FIG. 17b. In order to withdraw the retractor assembly, the actuator ring 168 is again pressed downwardly to deflect the legs 164 inwardly so that they can be withdrawn through the cannula. The retractor assembly 162 can be made of a shape memory alloy or of any suitable polymeric material. While the actuating surfaces 165 are shown as solid protrusions on the legs 164, they could also be formed by a suitable bend at a proximal end of the legs 164.

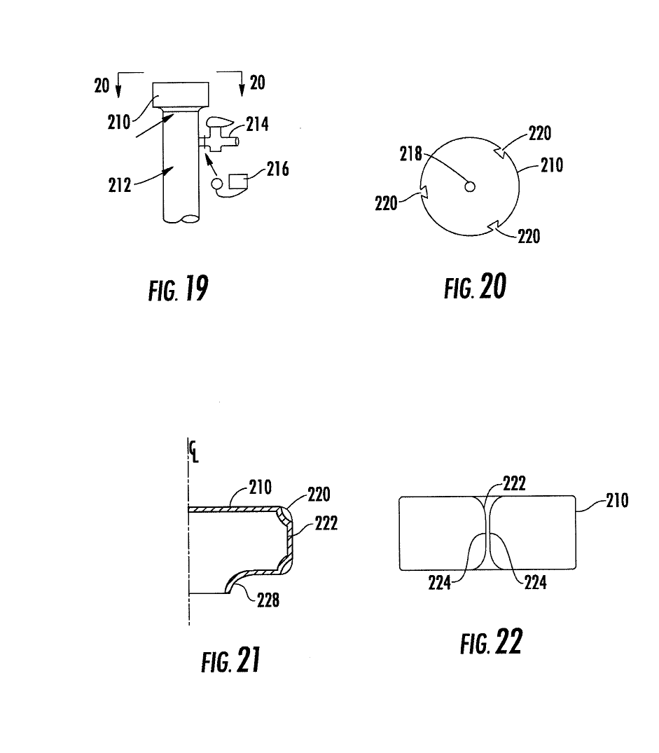

Referring now to FIGS. 19-22, a cap 210 for use in connection with the proximal end of a cannula 212 is shown. The cannula 212 is illustrated with a removable stopcock 214 and an optional cap 216 that can be connected to the stopcock or in place without it. However, those skilled in the art will recognize that the cap 210 can be used with multiple different configurations of the cannula 210, and that the illustrated embodiment is merely for illustrative purposes only. As shown in FIG. 20, the cap 210 preferably has a drip proof opening 218 located generally at the center thereof. Additionally, as shown in FIGS. 20-22, at least one, and preferably a plurality of channels 220 are located on the sidewalls of the cap 210 which can be used to attach sutures or other materials to the cap 210. Preferably, the channels 220 are formed with a recess or undercut as illustrated in FIGS. 21 and 22 so that the suture passes through a narrow opening 222, between the edges 224 defining the channel 220, which opens into a larger area which is undercut from the edges 224 so that the sutures or other material can be anchored to the cap 210.

As shown in FIGS. 19 and 21, preferably the bottom 228 of the cap provides a drip proof seal to the underside of the top portion of the cannula 212. This is preferably formed by using a sufficiently elastic type sealing material connected to the bottom 228 of the cap 210 or through a separate seal or bladder located inside the bottom edge of the cap 210.

The cap 210 can be used with various configurations of cannulas and different sizes can be provided in order to fit the proximal ends of different cannulas. By providing the drip proof opening through which instruments can be inserted in a sealing manner along with the channels for holding sutures to allow a cannula to be anchored firmly in position provides multiple advantages not currently available on existing cannulas.

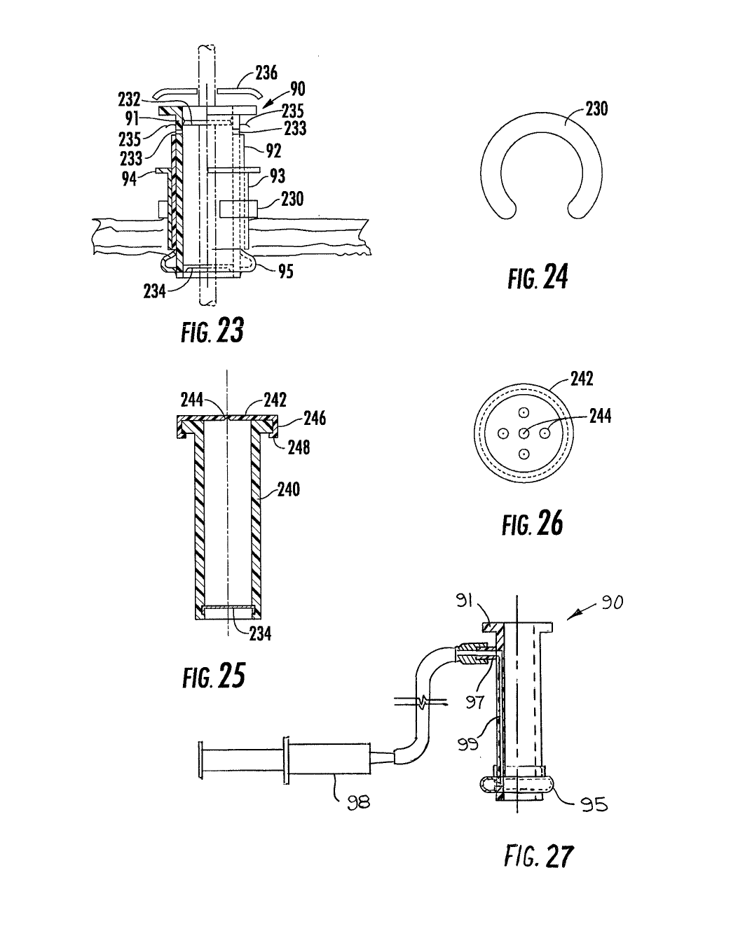

Referring to FIG. 23, the ninth embodiment of the cannula 90 is shown in a similar manner to FIG. 13, in which the cannula 90 is shown in cross-section on a left side of the centerline, but in an in-use position. The expanded projecting and sealing surface 95, formed by the bladder or elastic tube 92 that is attached to the distal end of the cannula and extends up the cannula body 91, is shown in the deployed state in order to anchor the cannula 90 in position. Additionally, an external C-shaped clamp 230, formed of a flexible polymeric material, is snapped onto the cannula body 91 and/or the tube 92 just above the outer surface of the tissue being penetrated, to prevent the cannula 90 from being unintentionally displaced further inwardly once it is set in position. The preferred clamp 230 is shown in detail in FIG. 24, and has an open portion between the ends of the C-shape through which the body 91 is received prior to being engaged. The distance between the ends of the C-shape is approximately 90% or less of the diameter of the body 91. While the clamp 230 is preferably flexible and can be snapped firmly into position, it is also possible to use a split clamping ring that is tightened onto the body 91 and/or elastic tube 92 using a hand screw or over-center toggle clamp type mechanism.

Still with reference to FIG. 23, additional baffles or seals 232, 234 are installed inside the body 91 of the cannula 90 at or near the distal and/or proximal ends, and/or anywhere therebetween. While a single seal 232, 234 is shown at the distal end and at the proximal end, those skilled in the art will understand that a single one of the seals 232, 234 could be provided at just the distal end or just the proximal end, or multiple seals can be provided at one or both ends or therebetween. The seals 232, 234 are formed as a diaphragm of an elastic polymeric material, and are pierceable by an instrument that is to be inserted through the cannula 90, and are resiliently deflectable, so that as the instrument, shown in phantom lines in FIG. 23, is manipulated, the seal 232, 234 is at least partially, and preferably fully maintained so that fluids from within a body cavity of the patient do not shoot or spray outwardly back through the cannula 90. The seals 232, 234 can be pressed into the cannula body 91 after installation in a patient and after removal of the trocar by mounting the seals 232, 234 on a sleeve shaped body that is pressed into the body 91, or by inserting and affixing one or more individual seals 232, 234 using appropriately shaped internal circular grooves in the body 91 which receive a peripheral edge of each seal 232, 234 and hold it in position.

Still with reference to FIG. 23, openings 233 can optionally be provided below the proximal seal 232 so that any pressurized fluid in the cannula is discharged sideways. Appropriate baffles 235 can be provided outside of the openings 233 to deflect the fluid back toward the patient's body. The baffles 235 can be made of a flexible elastomeric material and are preferable located above the openings 233.

Further, as shown in FIG. 23, a washer shaped baffle 236 can be attached to the proximal end of the cannula, or can be free standing and placed over the portion of the instrument being inserted so that any fluid that is expressed outwardly through the cannula 90 and past one or both seals 232, 234, is deflected and dispersed outwardly and downwardly, so that it does not strike the operator or assistant.

Referring to FIG. 25, a cannula body 240 is shown having a sealing cap 242, which can be snapped over the proximal end of the cannula 240. The cap 242 is made of a suitable elastomer, and preferably includes one or more defined weak points 244, as shown in FIG. 26, for easier insertion of a medical instrument through the cap 242. The cap includes a rim 246 and an inwardly directed bottom flange 248 to engage under the shoulder at the proximal end of the cannula 240.

Additionally, as shown in FIG. 25, a distal seal 234 can be provided as discussed above.

Generally referring to any of the above embodiments, the actuating sleeve can be formed from a singular piece of material or can be comprised of multiple materials in various segments along its length. The use of differing materials may be desired to produce optimal structural and deformation characteristics in the various embodiments. The sleeve may also feature stiffening elements used in conjunction with the fold seams to ensure formation and consistency with respect to the projecting surface(s) as well as to provide sufficient stiffness in the projecting surface(s) to facilitate use as a retractor.

The projecting surface(s) can take on any number of forms in addition to those described. The thickness, shape, and size may all be varied depending on the requirements of the application to facilitate improved sealing, retraction, ease of insertion or any number of other factors. The projecting surface(s) may be produced from any suitable material capable of withstanding the repeated deformation resulting from the contracting and expanding of the projecting surface(s).

The stop can be pre-formed in or on the body of an instrument, or can be a separate element attached to the body made from plastic, rubber, surgical steel, or any other material suitable for use in medical procedures. The stop can be attached by adhesives, fasteners, press fits, snap rings, and other mechanical means. Further, it is envisioned that the stop can be incorporated into an inner sleeve that extends beneath an outer sleeve that can be attached to an instrument body at a point located at a proximate end of an instrument body for easy access and position adjustment. This embodiment would eliminate any design challenge associated with fixing the stop to a point that is located in the surgical cavity. The stop may also be movable along the length of the instrument body in order to facilitate adjustment of the depth of insertion.

Referring to FIG. 27, which is shown in cross-section on the left side of the centerline, an alternate arrangement of the ninth embodiment of the invention is shown with the body 91 of the cannula 90 having a port 97 for connection to an outside source of pressurized fluid or air, such as the syringe 98, pressurized capsule or other pressurized source. In this case, the bladder 92 is positioned at the distal end of the body 91, and fluid is delivered from the syringe 98 through the port 97 and travels in a passageway 99 in the cannula body 91 to an area inside the bladder 92 in order to expand the bladder 92. Opening the port 97 allows the bladder 92 to return to its original position generally adjacent to the body 91 for easy insertion and removal.

With respect to one preferred application of the invention for cannulas, those skilled in the art will understand that the cannula body can have any shape or form, and the outer surface, or portions thereof can have a contoured surface to enhance holding in surrounding tissue. Additionally, the distal end of the cannula tubes are preferably provided without additional downwardly directed protrusions or walls that limit the field of view for an endoscope inserted through the cannula.

All of the above embodiments and various combinations thereof can be used with various surgical or non-surgical instruments for differing applications. They can be used in connection with solid tools to form a deployable retractor-type instrument, as well as with hollow instruments where further tools or instruments can then be passed through the hollow opening. Using the present invention, it is now possible to provide better holding of a cannula or other surgical instrument in position, as well as to provide a better seal against the tissue inside the incision in order to prevent additional swelling in fluid filled surgical sites, for example in arthroscopic/endoscopic knee or shoulder surgery. The invention also allows a cannula or endoscope, or other surgical instrument to be used as a retractor, which can allow a surgeon to increase a tissue limited field of view, which can be as little as 3 to 4 mm in arthroscopic surgery, to 20 mm or more through the ability to retract the tissue using the retractor capability of the instruments configured according to the invention. Use in connection with non-surgical instruments is also possible.

While several preferred embodiments have been disclosed, those skilled in the art will recognize that various portions of the embodiments could be combined with other ones of the embodiments to improve functionality. For example, pull wires could be used to stow or deploy various ones of the projecting surfaces in any of the above embodiments. Additionally, various ones of the locking mechanisms described above or other suitable locking mechanisms can be utilized as required for a particular application.

Having thus described in detail several embodiments of the present invention, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

What is claimed is:

1. A system for sealing and affixing an instrument body within an opening comprising:
a body having proximal and distal ends;
an expandable portion that increases in effective size to form a projecting surface in proximity to a distal end of the body.

2. The system of claim 1, further comprising an outer sleeve located on the body that is moveable between an installation state and a deployed state, in the installation state the projecting surface is in a retracted position against the body and in the deployed state the projecting surface extends away form the body.

3. The system of claim 2, wherein the projecting surface is annular.

4. The system of claim 2, wherein the projecting surface comprises a plurality of fingers.

5. The system of claim 2, wherein a distal end of the sleeve is fixed proximate to the distal end of the body.

6. The system of claim 2, wherein the body has a stop attached in proximity to the distal end.

7. The system of claim 2, wherein the sleeve has at least one folding line formed therein.

8. The system of claim 2, wherein the sleeve is lockable in at least one position relative to the body.

9. The system of claim 2, wherein the sleeve further comprises a handle.

10. The system of claim 9, wherein the handle is annular.

11. The system of claim 2, wherein the body is connected to a cannula.

12. The system of claim 2, wherein the body is connected to an endoscope.

13. The system of claim 2, wherein the body is connected to a surgical or non-surgical instrument or tool.

14. The system of claim 1, further comprising a seal located in the body.

15. The system of claim 1, further comprising a pierceable sealing cap attached to a proximal end of the body.

16. The system of claim 1, further comprising an outer clamp that is connected to the body.

17. The system of claim 1, further comprising a seal located at a proximal end of the body, and openings through a sidewall of the body located distal to the seal.

18. The system of claim 1, further comprising a baffle located at or above a proximal opening of the body.

19. A method of using an instrument inserted into a surgical site comprising the steps of:
providing an instrument to be inserted into a surgical opening having a stop attached thereto with a sleeve, the sleeve having an installation and deployed state, in the installation state the projecting surface is in a retracted position against the body and in the deployed state the projecting surface extends away form the body;
inserting the instrument and sleeve through tissue into a surgical site;
sliding the sleeve against the stop thereby altering the sleeve profile to the deployed state.

20. The method according to claim 19, further comprising:
pulling on the instrument as a retractor to provide additional space for surgical operations.

21. A method of using an instrument inserted into a surgical site comprising the steps of:
providing an instrument with first and second end to be inserted into a surgical site;
providing a sleeve, the sleeve having an alterable profile with a projecting surface pre-formed therein,
placing the sleeve over the instrument;
attaching the sleeve proximate to the first end to the instrument;
placing tension on the sleeve so as alter its profile to a generally cylindrical form;
inserting the instrument and sleeve into a surgical site;
releasing the sleeve thereby allowing the sealing surface to reform inside the bodily cavity.

22. The method according to claim 21, further comprising:
using the instrument as a retractor to provide additional space for further surgical operations.

23. A system for sealing and affixing a body within an opening comprising:
an elongated body having proximal and distal ends;
the body having at least one projecting surface attached thereto;
the projecting surface being movable between a stowed and a deployed state so as to form a projecting surface in proximity to a distal end of the body.

24. The system of claim 23, wherein the projecting surface is deployed by a handle.

25. The system of claim 23, wherein the projecting surface is deployed by a rotatable collar located near the proximal end of the body.

26. The system of claim 23, wherein the sealing surface is an inflatable bladder.

27. A method for sealing and affixing a body within an opening comprising:
providing an elongated body having proximal and distal ends;
the body having at least one projecting surface in contact therewith;
inserting the body into the opening and deploying the projecting surface in proximity to a distal end of the body.

28. A cap for sealing an end of a cannula, comprising:
an upper surface with a drip-proof opening for insertion of an instrument therethrough located in a top thereof;
a side wall with at least one channel having an undercut section; and
a sealing lip located along a bottom edge of the side wall.
